(19) United States
(12) Patent Application Publication
(10) Pub. No.: US 2015/0147775 A1
(43) Pub. Date: May 28, 2015

(54) TEST METHOD FOR EFFICACY OF SILVER SURFACES AS A SANITIZER

(71) Applicant: CORNING INCORPORATED, CORNING, NY (US)

(72) Inventors: Derek Michael Fiacco, Corning, NY (US); Jeffrey Glenn Lynn, Tioga, PA (US); Kaitlyn Mary Matias, Painted Post, NY (US); Florence Christine Monique Verrier, Corning, NY (US); Ying Wei, Painted Post, NY (US)

(21) Appl. No.: 14/547,487

(22) Filed: Nov. 19, 2014

Related U.S. Application Data
(60) Provisional application No. 61/908,401, filed on Nov. 25, 2013.

(51) Int. Cl. C12Q 1/20; C12N 1/20

(52) U.S. Cl. CPC C12Q 1/20; C12N 1/20; G01N 2333/31; G01N 2333/265; G01N 2333/21; G01N 2333/245
USPC 435/33; 435/252.4

(57) ABSTRACT

A method that includes the steps: inoculating nutrient agar with bacterial stock to form a culture; incubating the culture to form a first incubated culture; incubating a portion of the first culture with nutrient agar to form a second culture; incubating a portion of the second culture to form a third culture; incubating the third culture to form an inoculated test plate; forming an inoculum by suspending bacteria from the inoculated test plate in a buffered test solution, adjusting the pH to ˜7 to 8, and adding organic soil at a concentration of approximately 10% to 30% by weight; inoculating a silver-containing surface region of a test carrier with a portion of the inoculum; incubating the inoculated test carrier; washing the test carrier in a neutralizing solution to form a residual test inoculum; and calculating the percent reduction in the number of surviving bacterial colonies in the residual test inoculum.

This application claims the benefit of priority under 35 U.S.C. §119 of U.S. Provisional Application Ser. No. 61/908,401 filed on Nov. 25, 2013 the content of which is relied upon and incorporated herein by reference in its entirety.

### TECHNICAL FIELD

The present disclosure relates generally to methods of testing the antimicrobial efficacy of silver-containing surfaces including the composition and preparation of inoculums for such testing.

### BACKGROUND

Various health benefits associated with metallic silver have been developed over the last few hundred years. Various antiseptic, antibacterial, antiviral and other antimicrobial effects have been observed and documented associated with the use of silver in various articles. Silver vessels, for example, were used in the care of ill and wounded individuals in the Middle Ages. More recently, the incorporation of metallic silver particles and silver salts into a variety of materials including yarns, fabrics, and glasses has been described as a means of imparting antibacterial properties to articles containing these materials. Silver ions, for example, can be incorporated into the surface of prosthetic devices having glass, glass-ceramic or ceramic surfaces to impart these surfaces with antimicrobial properties.

The U.S. Food and Drug Administration approved silver solutions as antibacterial agents in the 1920s. However, only recently scientists have begun to explore how, why and where silver works as an antimicrobial agent. Silver has been shown to have antibacterial, antifungal, antiviral, anti-inflammatory, antibiofilm properties. Many gram-negative bacteria such as Acinetobacter, Escherichia, Pseudomonas, Salmonella, Vibrio and gram-positive bacteria including Bacillus, Clostridium, Enterococcus, Listeria, Staphylococcus, and Streptococcus are sensitive to silver. Silver is a fast-acting fungicide against a broad spectrum of common fungi including genera such as Aspergillus, Candida and Saccharomyces. Silver has also been demonstrated to be effective against viruses such as Human Immuno Deficiency Virus (HIV-1). To date, silver ions are known to be effective against over 650 types of bacteria.

With the wide use of antibiotics in today's world, antibiotic-resistant bacteria such as methicillin-resistant Staphylococcus aureus (MRSA), vancomycin-resistant Enterrococcus faecium, etc. are commonly associated with nosocomial infections (i.e., infections resulting from receiving treatment in a healthcare service unit, but secondary to a patient's original condition). Such antibiotic resistant microbes are also very sensitive to the biocidal effect of silver. With the increasing cost of Hospital Acquired Infections (HAIs), antimicrobial intervention strategies to reduce contaminated surfaces and reduce incidence of cross contamination has become a major focus in the health care industry.

In contemporary society, computerized and electronic “touch screens” are prevalent in various consumer products, e.g., automatic teller machines, gasoline pumps, mobile phones, etc. Generally, these touch screens possess glass, glass-ceramic and ceramic substrates. Recent studies suggest that touch screens harbor large quantities of microbes, bacteria and viruses harmful to humans. Recent developments indicate that silver ions incorporated into the substrates of these touch screens can impart antimicrobial properties to these products.

In the United States, antimicrobial products are regulated as pesticides by the U.S. Environmental Protection Agency (“EPA”). To be registered with public health claims (stating protection for the user from bacteria or other microbial organisms that can lead to health impact), antimicrobial products must demonstrate antimicrobial efficacy using recommended test methods that are either a controlled in-use study or simulated in-use study.

So far, the only test method using a simulated in use study and being approved by the EPA for public health claim, is the Test Method for Efficacy of Copper Alloy Surfaces as a Sanitizer (“Copper Test Protocol”). Although this method has become the reference method, there are still a number of significant scientific challenges in establishing valid test methods, and performance standards for product claims specific for the product end use (e.g., touch screen applications). Moreover, this standard method has been successfully used to assess the efficacy of copper alloys, but has failed to measure the efficacy of other antimicrobial technologies.

No EPA-approved test methods exist today for surfaces incorporated with silver ions as an antimicrobial product. Particular methods are not available to characterize the efficacy of glasses, glass-ceramic, ceramic substrates and other surfaces incorporated with silver ions as antimicrobial products. Furthermore, data suggests that the Copper Test Protocol is not capable of measuring with robustness the antimicrobial efficacy of surfaces containing silver ions. There is therefore a need to develop a protocol suitable for testing the antimicrobial efficacy of surfaces containing silver ions.

### SUMMARY

According to a first embodiment, a method for testing the anti-microbial efficacy of a silver-containing surface region is provided. The method includes the steps: inoculating nutrient agar with a portion of a stock having a plurality of bacterial organisms to form a culture; incubating the culture to form a first incubated culture; incubating a portion of the first incubated culture with nutrient agar to form a second incubated culture; incubating a portion of the second incubated culture to form a third incubated culture; and incubating the third incubated culture for approximately 48 hours to form an inoculated test plate with a plurality of bacterial colonies. The method also includes the steps: forming an inoculum by suspending a portion of the plurality of bacterial colonies in a buffered test solution, adjusting the test solution to a pH of approximately 7 to 8, and adding an organic soil serum at a concentration of approximately 10% to 30% by weight to the test solution; inoculating a silver-containing surface region of a test carrier with a portion of the inoculum; and incubating the inoculated test carrier for at least approximately two hours. The method further includes the steps: washing the incubated and inoculated test carrier in a neutralizing solution to form a residual test inoculum; counting the number of surviving bacterial colonies per volume in the residual test inoculum; and calculating the percent reduction in the number of surviving bacterial colonies in the residual test inoculum relative to a residual control inoculum.

According to a second embodiment, a method for testing the anti-microbial efficacy of a silver-containing surface region is provided. The method includes the steps: inoculating nutrient agar with a portion of a stock having a plurality of bacterial organisms to form a culture; incubating the culture to form a first incubated culture; incubating a portion of the first incubated culture with nutrient agar to form a second incubated culture; incubating a portion of the second incubated culture to form a third incubated culture; and incubating the third incubated culture for approximately 48 hours to form an inoculated test plate with a plurality of bacterial colonies. The method also includes the steps: forming an inoculum by suspending a portion of the plurality of bacterial colonies in a buffered test solution, adjusting the test solution to a pH of approximately 7 to 8, and adding an organic soil serum at a concentration of approximately 10% to 30% by weight to the test solution; inoculating a silver-containing surface region of a test carrier comprising an inorganic glass material with a portion of the inoculum; and incubating the inoculated test carrier for at least approximately two hours. The method further includes the steps: washing the incubated and inoculated test carrier in a neutralizing solution to form a residual test inoculum; counting the number of surviving bacterial colonies per volume in the portion of the residual test inoculum; and calculating the percent reduction in the number surviving bacterial colonies in the portion of the residual test inoculum relative to a residual control inoculum. In addition, the plurality of bacterial organisms is selected from one of the group consisting of Staphylococcus aureus, Enterobacter aerogenes, Pseudomonas aeruginosa and Escherichia coli.

According to a third embodiment, a method of preparing an inoculum for testing the anti-microbial efficacy of a silver-containing surface region is provided. The method includes the steps: inoculating nutrient agar with a portion of a stock having a plurality of bacterial organisms to form a culture; incubating the culture to form a first incubated culture; incubating a portion of the first incubated culture with nutrient agar to form a second incubated culture; incubating a portion of the second incubated culture to form a third incubated culture; and incubating the third incubated culture for approximately 48 hours to form an inoculated test plate with a plurality of bacterial colonies. The method also includes the step: forming an inoculum by suspending a portion of the plurality of bacterial colonies in a buffered test solution, adjusting the test solution to a pH of approximately 7 to 8 and adding an organic soil serum at a concentration of approximately 10% to 30% by weight to the test solution. Further, the plurality of bacterial organisms is selected from one of the group consisting of Staphylococcus aureus, Enterobacter aerogenes, Pseudomonas aeruginosa and Escherichia coli.

According to a fourth embodiment, an inoculum for testing the anti-microbial efficacy of a silver-containing surface region is provided. The inoculum includes an inoculum comprising (a) a plurality of bacterial colonies; (b) an organic soil serum at a concentration of approximately 10% to 15% by weight; and (c) a buffering solution. Further, the inoculum has a pH of approximately 7 to 8.

According to a fifth embodiment, an antimicrobial glass is provided. The antimicrobial glass includes a glass substrate having a silver-containing surface region. The surface region is characterized by a log kill rate of 2 or greater as tested by the inoculum of the fourth embodiment.

According to a sixth embodiment, an antimicrobial glass is provided. The antimicrobial glass includes a glass substrate having a silver-containing surface region. The surface region is characterized by a log kill rate of 2 or greater as tested by the method of the first embodiment.

According to a seventh embodiment, an antimicrobial glass is provided. The antimicrobial glass includes a glass substrate having a silver-containing surface region. The surface region is characterized by a log kill rate of 2 or greater as tested by the method of the second embodiment.

According to an eighth embodiment, an antimicrobial glass is provided. The antimicrobial glass includes a glass substrate having a silver-containing surface region. The surface region is characterized by a log kill rate of 2 or greater as tested by the method of the third embodiment.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

FIG. 1A is schematic depicting the steps involved in the Copper Test Protocol.

FIG. 1B is a schematic depicting the contents of the inoculum employed in the Copper Test Protocol.

FIG. 2 is a schematic depicting the effects of modifications to the inoculum employed in the Copper Test Protocol when used to test the antimicrobial efficacy of a glass surface incorporating silver ions.

FIG. 3 is a bar graph depicting the log kill rate versus inoculum pH level for inoculum formulations employed to test the antimicrobial efficacy of a glass surface incorporating silver ions according to one embodiment.

FIG. 4 is schematic depicting the steps involved in a protocol for testing the antimicrobial efficacy of the surface region of an article having silver ions according to another embodiment.

FIG. 5 is a bar graph depicting the log kill rate versus various inoculum preparations employed to test the antimicrobial efficacy of a glass surface incorporating silver ions according to an additional embodiment.

FIG. 6 is a bar graph depicting the log kill rate versus the concentration of sodium bicarbonate in a Hank's Balanced Salt Solution (“HBSS”) employed to test the antimicrobial efficacy of a glass surface incorporating silver ions according to a further embodiment.

FIG. 7 is a plot of the log kill rate versus the concentration of organic soil in an inoculum buffered with a Phosphate Buffered Solution (“PBS”) employed to test the antimicrobial efficacy of a glass surface incorporating silver ions according to an embodiment.

### DETAILED DESCRIPTION

Reference will now be made in detail to the present preferred embodiments, examples of which are illustrated in the accompanying drawings. Whenever possible, the same reference numerals will be used throughout the drawings to refer to the same or like parts.

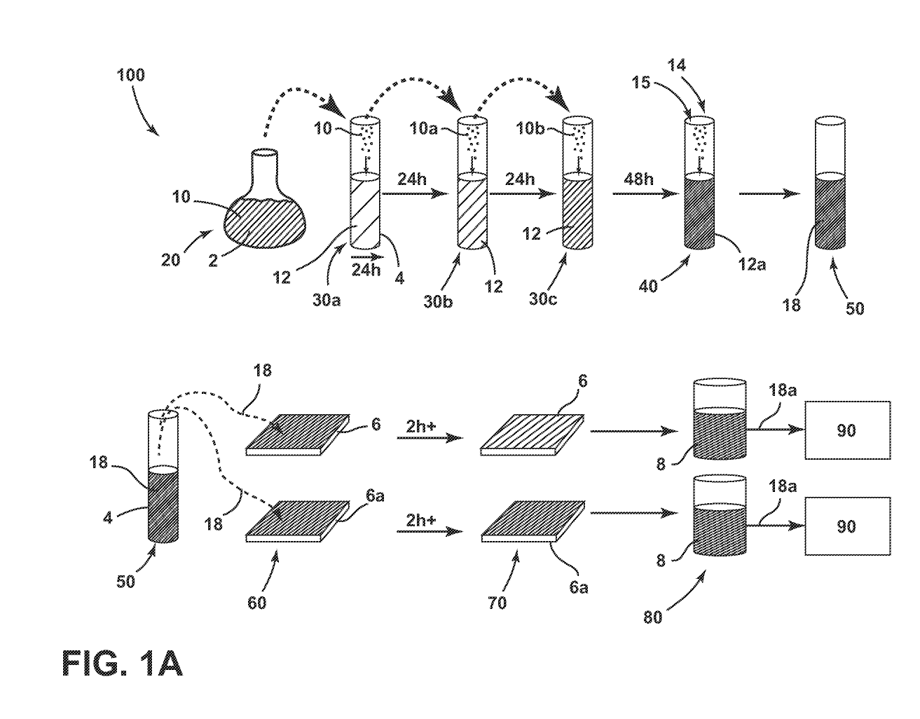

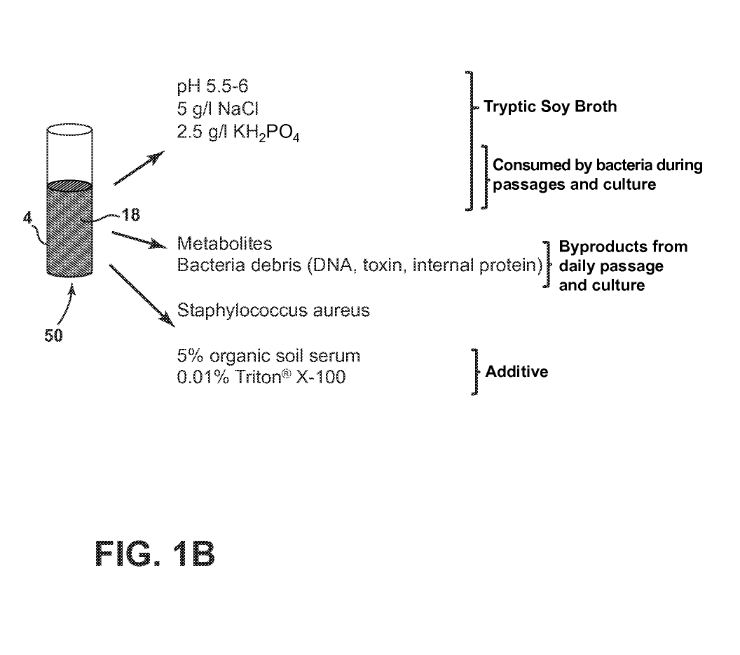

The Copper Test Protocol is incorporated by reference herein. As schematically depicted in FIG. 1A, a copper test protocol 100 that follows the Copper Test Protocol can be used to test the antimicrobial efficacy of copper containing surfaces. In general, copper test protocol 100 includes steps 20 through 50 to prepare an inoculum 18. Inoculum 18 is then employed to evaluate the antimicrobial efficacy of a copper-containing test carrier 6 relative to a control test carrier 6a in steps 60 through 90.

In step 20, a tester obtains a stock 2 of bacterial organisms 10. Bacterial organisms 10 can include any one of colonies of Staphylococcus aureus, Enterobacter aerogenes, Pseudomona aeruginosa, and Escherichia coli for a given test sequence. Once the microorganism strain is selected for the bacterial organisms 10, a tube 4 (or other suitable container) containing a broth 12 is inoculated with a portion of the bacterial organisms 10 in step 30a. The broth 12 may consist of a Tryptic Soy Broth (“TSB”) formulation as understood by those with ordinary skill in the field. The inoculated broth 12 in step 30a is then incubated for 24±2 hours at 35-37° C.

In step 30b, a portion of bacterial organisms 10a obtained from step 30a (incubated for 24±2 hours) is introduced into a fresh broth 12. The now-inoculated broth 12 with bacterial organisms 10a in step 30b is then incubated for 24±2 hours at 35-37° C. Finally, in step 30c, a portion of the bacterial organisms 10b obtained from step 30b (incubated for 48±4 hours) is introduced into a fresh broth 12. The inoculated broth 12 in step 30c with bacterial organisms 10b is then incubated for 24±2 hours at 35-37° C., thus forming an inoculated test broth 12a (see step 40).

In step 40, the inoculated test broth 12a is incubated for an additional 48±4 hours at 35-37° C. Organic soil 14 and surfactant 15 are then added to the inoculated test broth 12a. For example, the organic soil 14 can be fetal bovine serum and a TRITON® X-100 formulation can be used as the surfactant 15 as readily understood by those with ordinary skill in the field. Preferable, the organic soil 14 and surfactant 15 are added at concentrations of 5% and 0.01% by weight, respectively. An inoculum 18 is thus formed from the inoculated test broth 12a as depicted in step 50 in FIG. 1A.

Inoculum 18 can then be employed to test the antimicrobial efficacy of copper-containing test carrier 6 and control test carrier 6a in steps 60 and 70. In step 60, a portion of the inoculum 18 is spread on copper-containing test carrier 6 and control test carrier 6a and dried. Under the copper test protocol 100, test carrier 6 comprises a copper alloy and control test carrier 6a comprises a stainless steel. In step 70, the dried portion of inoculum 18 is exposed on the test carriers 6, 6a for about 120 minutes.

Next, in step 80, the exposed test carriers 6 and 6a are transferred separately into a neutralizer solution 8 and sonicated to obtain a residual test inoculum 18a. The number of surviving bacterial colonies from the residual test inoculum 18a associated with each of the test carriers 6 and 6a is then counted by standard techniques. For example, the residual test inoculum 18a can be spread on a Tryptic Soy Agar (“TSA”) plate or a 5% sheep Blood Agar Plate (“BAP”) for purposes of bacterial colony counting. The acceptance criterion for copper protocol 100 includes an assurance that the minimum bacterial recovery on the control test carrier 6a (without Cu) is equivalent to 2×10⁴ CFU/carrier (see Equation (1) below).

Finally, in step 90, various calculations can be conducted using the raw data obtained from step 80 associated with copper-containing test carrier 6 and control test carrier 6a. For instance, the percent reduction in the number of surviving bacterial colonies per volume in the residual test inoculum 18a associated with each of the copper-containing test carriers 6 can be calculated according to standard methods as commonly understood in the art. Equations (1) through (5) below can be used to calculate such percent reduction values associated with each bacterial microorganism 10 tested on copper-containing test carrier 6 and control test carrier 6a.

In Equation (1) below, the number of bacterial microorganism 10 colonies can be calculated in terms of colony forming units per carrier (CFU) as follows:

CFU/carrier=(x_{colonies}/agar plate)×(dilution)×(vol_{neutralizer solution)}/vol_{plated} (1)

where x_{colonies}/agar plate is the average number of bacterial colonies counted on each agar plate, dilution is the dilution factor, vol_{neutralizer} solution is the volume of neutralizer solution used in the testing and vol_{plated} is the volume of material plated on the agar plates. As such, Equation (1) can be used to calculate the CFU/carrier values associated with the copper-containing test carrier 6 and control test carrier 6a.

In Equation (2), the geometric mean number of surviving bacterial microorganisms 10 can be calculated for the copper-containing test carrier 6 as follows:

$${mean}_{control test carrier} = antilog of \frac{log {Y}_{1} + log {Y}_{2} + log {Y}_{N}}{N} \qquad ( 3 )$$

where Y₁, Y₂, Y₃, etc. are the CFU/carrier values for each successively tested copper-containing test carrier 6, and N relates to the number of such tests. Typically, N is set to 5 tests for the copper-containing test carrier 6.

In Equation (3), the geometric mean number of surviving bacterial microorganisms 10 can be calculated for the control test carrier 6a as follows:

$${mean}_{test carrier} = antilog of \frac{log {Y}_{1} + log {Y}_{2} + log {Y}_{3} + log {Y}_{4} + log {Y}_{N}}{N} \qquad ( 2 )$$

where X₁, X₂, etc. are the CFU/carrier values for each successively tested control test carrier 6a, and N relates to the number of such tests. Typically, N is set to 3 tests for the control test carrier 6a.

Equation (4) below relies on the data generated in Equations (2) and (3) and provides the percent reduction in bacterial organisms tested on a given copper-containing test carrier 6:

% reduction=[(mean_{control test carrier}−mean_{test carrier})/mean_{control test carrier}]×100 (4)

where the mean_{control test earner} is obtained from Equation (2) and the mean_{test carrier} is obtained from Equation (3). As such, the % reduction value reflects the relative degree of bacterial killing or anti-microbial efficacy of a copper-containing test carrier 6 relative to a control surface, e.g., test control carrier 6a.

It should also be understood that a “log kill” relates to the % reduction obtained in Equation (4) according to the following relation given by Equation (5) below:

log kill=−(log(1−% reduction)) (5)

As such, a 99% reduction in bacterial organisms for a copper-containing test carrier 6 is equivalent to a log kill of 2.0.

The Copper Test Protocol can be employed to test the anti-microbial efficacy of a copper-containing surface to support claims that the tested surface kills greater than 99.9% of the particular bacterial organism tested. However, recent work by the named inventors suggest that the Copper Test Protocol is not effective at assessing the anti-microbial efficacy of silver-containing surfaces, such as glass sheet and films having a surface region containing silver ions. For example, Corning Ag IX glass having a surface region with silver ions was tested with the Copper Test Protocol. In these tests, % reduction levels against S. aureus was only approximately 70 to 80% or a log kill of approximately 0.5 to 0.7. In essence, the Copper Test Protocol cannot be used to readily ascertain the anti-microbial efficacy of surfaces containing silver ions relative to control surfaces.

Some understanding of how silver ions function with an antimicrobial effect is necessary to develop new protocols for assessing the antimicrobial efficacy of silver-containing surfaces. Different classes of bacteria have different membrane structures. These membranes may contain peptidoglycan layers in addition to phospholipids and lipopolysaccharides outer layers. It is believed that small silver ions can associate with and penetrate membranes causing a structural change in the membrane. This causes increased cell permeability, and transport of silver through the inner cytoplasmic membrane. Silver ions act as antimicrobial agents by strongly binding to critical biological molecules (proteins, DNA, RNA) and disrupting their function(s). Silver chelates with thiol groups in proteins (containing cysteine amino acids) disrupting the activity of vital enzymes critical to cellular signaling needed for bacterial growth. In addition, silver complexes with the adenine, and guanine bases in nucleic acid (DNA and RNA). This causes disruption of DNA replication and cell division (bacteriostatic effect) ultimately leading to metabolite efflux resulting in cell death (bactericidal effect). Ultimately the modes of action depend on the concentration of silver ions present and the sensitivity of the microbial species to the silver ions. Contact time, temperature, pH and the presence of free water likewise impact the rate and extent of antimicrobial activity afforded by the silver ions.

Disclosed herein are embodiments of methods of testing the antimicrobial efficacy of silver-containing surfaces, including the composition and preparation of inoculums for such testing. These embodiments were developed in view of the foregoing silver-related antimicrobial mechanisms and to better mimic the end-use applications for silver-containing surfaces. These embodiments also hew to principles of the Copper Test Protocol where possible, recognizing that they are structured to be qualified as test protocols by the EPA. But the embodiments also significantly depart from the Copper Test Protocol in view of the particular testing environment presented by articles containing a silver-containing surface region and to better mimic in-use bacterial contamination (e.g., fingerprint fomites in touch screen applications).

As depicted in FIG. 1B, an inoculum 18 prepared in the copper test protocol 100 at step 50 contains various constituents and reflects significant changes from its initial formulation. For example, the glucose, casein peptone and soya peptone, originally present in the TSB employed in the broth 12, are not present at significant levels in the inoculum 18. This is because these sugars and proteins are consumed as food by the bacterial organisms 10, 10a and 10b added to the broth 12 during steps 30a-30c. At the same time, various metabolites and bacterial debris are formed in the inoculum as the bacterial organisms 10, 10a and 10b are cultured within the broth 12 in steps 30a through 40. The formation of these metabolites and bacterial debris, and consumption of the sugars and proteins, tends to reduce the pH of the inoculum to approximately 5.5 to 6. It is believed that the release of metabolites during bacterial growth inhibits the ability of the inoculum 18 derived from the copper test protocol 100 to perform as an agent for testing the anti-microbial efficacy of a silver-containing surface.

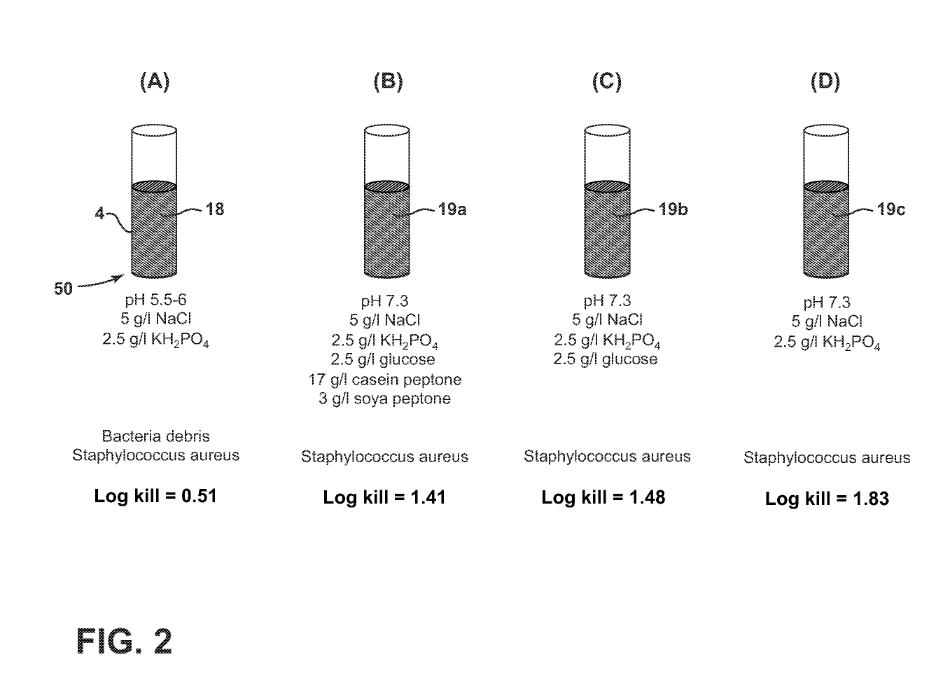

As depicted in FIG. 2, various modifications were made to the inoculum 18 obtained at step 50 in the copper test protocol 100 using Staphylococcus aureus as the bacterial organism 10 (see (A) in FIG. 2), thereby producing modified inoculums 19a, 19b and 19c (see (B)-(D) in FIG. 2). Inoculum 19a was prepared by removing the metabolites and bacterial debris present in inoculum 18. This was accomplished by centrifuging the live Staphylococcus bacterial organisms 10b in the inoculum 18 using commonly understood procedures in the art, followed by refreshing the broth with a further quantity of TSB. The resultant inoculum 19a is depicted at (B) and possesses a pH of approximately 7.3, significantly more neutral than the pH level of inoculum 18. Inoculum 19b was prepared in a process comparable to that used for inoculum 19a, except that its broth was refreshed with a salt solution having glucose and no protein constituents (e.g., casein and soya peptone). Inoculum 19c was also prepared in a process comparable to that used for inoculum 19a, except that its broth was refreshed with a salt solution having no glucose or protein constituents. Inoculums 19b and 19c both possess a pH of approximately 7.3.

These modifications depicted in FIG. 2 were made for the purpose of assessing the impact of changes to the formulation and processing of inoculum 18 with regard to testing the anti-microbial efficacy of an article containing a silver-containing surface region. As shown in FIG. 2, the inoculum 18 produced a log kill of 0.51 when tested on a glass test carrier having a surface region with silver ions. In comparison, inoculums 19a, 19b and 19c each had substantially higher log kill values, 1.41, 1.48 and 1.83, respectively. Based on the results from this experiment, it is believed that increasing the pH level of the inoculum will improve testing efficacy as evidenced by the increase in log kill values. It is also believed that bacterial debris in the inoculum may form chelates with the silver ions in the test carriers (e.g., test carrier 6), reducing the ability of the silver to kill or otherwise degrade the bacterial organisms 10b within inoculums 18, 19a, 19b, and 19c. As such, inoculum 19c with no sugar, proteins and bacterial debris demonstrated the highest log kill values in the experiment.

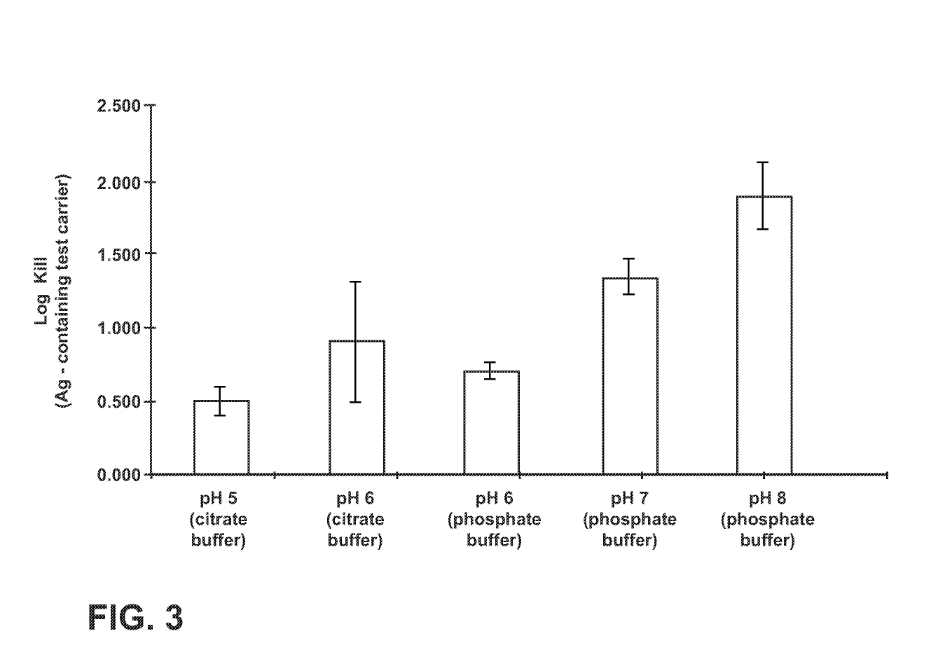

As depicted in FIG. 3, a further experiment was conducted to further assess the effect of inoculum pH level on log kill for a glass substrate having a surface region with silver ions. Various inoculums were prepared in the experiment comparable to the inoculum 19c depicted in FIG. 2, refreshed with buffered salt solutions to obtain five inoculums with a pH ranging from 5 to 8. Two solutions had a pH of 6, one with a citrate buffer and the other with a phosphate buffer. FIG. 3 demonstrates that only the inoculums having a pH of 7 to 8 exhibited log kill values greater than 1. As such, it is important to maintain a pH level close to physiological, neutral conditions to obtain high testing efficacy when evaluating the antimicrobial efficacy of silver-ion containing systems. It should also be noted that the log kill values near and below 1.0 for the inoculums with a pH of less than 7 are essentially noise. Hence, the results from the phosphate and citrate buffered inoculums having a pH of 6 shown in FIG. 3 are inconclusive.

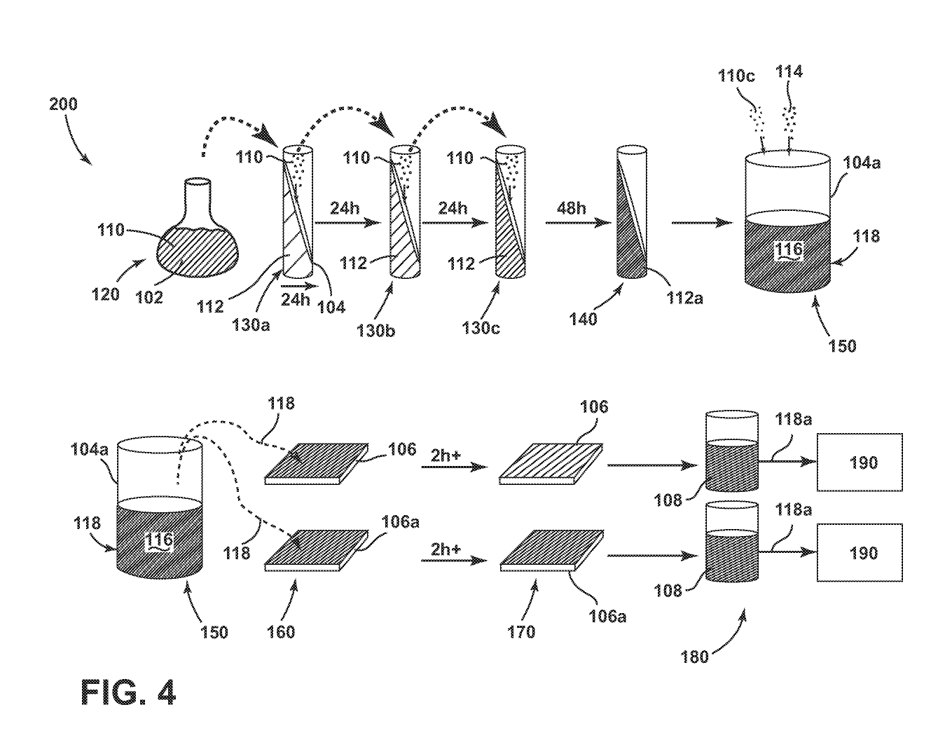

As schematically depicted in FIG. 4, the Ag Protocol 200 was developed in view of the experiments depicted in FIGS. 2 and 3, among other findings and studies. Ag Protocol 200 can be used to test the antimicrobial efficacy of silver-containing surfaces. In general, the Ag Protocol 200 includes steps 120 through 150 to prepare an inoculum 118. Inoculum 118 is then employed to evaluate the antimicrobial efficacy of an Ag-containing test carrier 106 relative to a control test carrier 106a in steps 160 through 190. The Ag-containing test carrier 106 can be any article having an exposed surface with a surface region containing Ag ions. Preferably, the article used for test carrier 106 is a 1 in.×1 in. square of high-strength substrate glass (e.g., a display device substrate glass strengthened through ion exchange processes), such as Corning Gorilla® glass, having a surface region containing Ag and alkali metal ions. Typically, the control test carrier 106a will be the same underlying substrate material of Ag-containing test carrier 106, but lacking Ag.

In step 120, a tester obtains a stock 102 of bacterial organisms 110. Bacterial organisms 110 can include any one of Staphylococcus aureus, Enterobacter aerogenes, Pseudomona aeruginosa, and Escherichia coli for a given test sequence. Once the microorganism strain is selected for the bacterial organisms 110, a tube 104 (or other suitable container, such as a plate) containing nutrient agar 112 is inoculated with a portion of the bacterial organisms 110 in step 130a. The nutrient agar 112 is a formulation for culturing bacterial organisms as understood by those with ordinary skill in the field. The inoculated nutrient agar 112 within tube 104 in step 130a is then incubated for 24±2 hours at 35-37° C.

In step 130b, a portion of bacterial organisms 110a obtained from step 130a (incubated 24±2 hours) is introduced into fresh nutrient agar 112. The inoculated nutrient agar 112 within tube 104 in step 130b is then incubated for 24±2 hours at 35-37° C. Finally, in step 130c, a portion of bacterial organisms 110b obtained from step 130b (incubated for 48±4 hours) is introduced into fresh nutrient agar 112. The inoculated nutrient agar 112 within tube 104 in step 130c (with bacterial organisms 110b) is then incubated for 24±2 hours at 35-37° C., thus forming a cultured nutrient agar 112a.

In step 140, the cultured nutrient agar 112a is incubated for an additional 48±4 hours at 35-37° C., thus developing bacterial colonies 110c. As described earlier, the cultured nutrient agar 112a may now exist in a slightly acidic condition (pH˜5.5 to 6).

In step 150, an inoculum 118 is formed by suspending a portion of the bacterial colonies 110c from the cultured nutrient agar 112a (obtained at step 140) in a buffered test solution 116 within container 104a. By collecting only a portion of the bacterial colonies 110c in the nutrient agar 112a, bacterial debris and metabolites are not placed into the buffered test solution 116. In addition, bacterial colonies 110c can be re-suspended directly in buffered test solution 116 to obtain a more physiological condition. Other approaches as understood by those with ordinary skill can also be used to adjust buffered test solution 116 to a pH of approximately 7 to 8.

Further, organic soil 114 is added to the buffered test solution 116 at a concentration of 10 to 30% by weight. Preferably, the organic soil is added at a concentration of 10 to 15% by weight. Further, the organic soil 114 may comprise fetal bovine serum. In the Ag Protocol 200 the organic soil 114 serves the same purpose as the soil 14 in the copper test protocol 100. That is, the soil 114 is incorporated into the solution to better mimic real-world conditions in which the article having the silver-containing surface region contains various soiling (e.g., fingerprint oils, mucus, blood, and other organic detritus). On the other hand, no surfactant (compare surfactant 16 in step 40 of the copper test protocol 100) is added in step 150 to the buffered test solution 116. Other data suggests that the use of a surfactant in the buffered test solution 116 and its presence in the inoculum 118 would tend to reduce the wettability of the inoculum 118 on test carriers 106, 106a when hydrophobic silver-containing surfaces are tested. Non-uniform spreading of the inoculum 118 on test carriers 106, 106a will result in non-robust measurements of the antimicrobial efficacy of the carrier 106.

Inoculum 118 can then be employed to test the antimicrobial efficacy of a silver-containing test carrier 106 and control test carrier 106a in steps 160 and 170. In step 160, a portion of the inoculum 118 is spread on a 1 in.×1 in. square of silver-containing test carrier 106 and a 1 in.×1 in. square of control test carrier 106a and dried. In the Ag Protocol 200, test carrier 106 comprises a silver-containing surface region and a substrate material, and control test carrier 106a comprises the substrate material (lacking Ag). In step 170, the dried portion of inoculum 118 is exposed on the test carriers 106 and 106a for at least about two hours. Preferably, the exposure of inoculum 118 on test carrier 106 and 106a is conducted for at least four hours.

Next, in step 180, the exposed test carriers 106 and 106a are transferred separately into a neutralizer solution 108 and are each sonicated to obtain a residual test inoculum 118a that corresponds to the carriers 106 and 106a. The number of surviving bacterial colonies from the residual test inoculum 118a associated with each of the test carriers 106, 106a is then counted by standard techniques. For example, the residual test inoculum 118a can be spread on a TSA plate or a 5% sheep BAP for purposes of bacterial colony counting. The acceptance criterion for Ag Protocol 200 is similar to the one described in the copper test protocol 100. That is, the minimum bacterial recovery on the control carriers (without Ag) should be equivalent to 2×10⁴ CFU/carrier.

Finally, in step 190, various calculations can be conducted using the raw data obtained from step 180 associated with silver-containing test carrier 106 and control test carrier 106a. For instance, the percent reduction in the number of surviving bacterial colonies per volume in the residual test inoculum 118a associated with each of the silver-containing test carriers 106 can be calculated according to standard methods as commonly understood in the art. Equations (1) through (5) above can be used to calculate such percent reduction values associated with each bacterial microorganism 110 tested on the silver-containing test carrier 106 and control test carrier 106a.

The test carrier 106 utilized in the Ag Protocol 200 is an article that possesses a silver-containing surface region (configured for anti-microbial effects). The test carrier 106 is preferably an inorganic glass, ceramic or glass-ceramic material having a silver-containing surface region. The test carrier 106 may also comprise a hydrophobic layer, such as a polymeric coating, over or under the silver-containing surface region. In test carrier 106 configurations with a hydrophobic layer beneath the silver-containing surface region, the substrate beneath the silver-containing surface region and the hydrophobic layer can comprise metals, composites, ceramics and/or polymeric materials.

The buffered test solution 116 employed at step 150 of the Ag Protocol 200 (see FIG. 4) can comprise various commercial formulations to achieve a neutral and stable pH. As outlined in Table 1 below, the buffered test solution 116 can comprise TSB (“Fresh TSB”), PBS, HBSS, a modified HBSS with no calcium and magnesium salts (“HBSS—no Ca/Mg”), and Eagle's Minimal Essential Medium (“EMEM”) solutions. For each of the buffered test solutions 116 listed in Table 1, the constituents reflect the condition (e.g., pH from approximately 7 to 8) of the solution at step 150.

TABLE 1

| Fresh TSB | | PBS | | HBSS | | HBSS - no Ca/Mg | | EMEM | |
|---|---|---|---|---|---|---|---|---|---|
| pH 7.3 | | pH 7.05 | | pH 7.75 | | pH 8.02 | | pH 7.40-7.90 | |
| 5 | g/l NaCl | 8 | g/l NaCl | 8 | g/l NaCl | 8 | g/l NaCl | 6.8 | g/l NaCl |
| | | 0.2 | g/l KCl | 0.40 | g/l KCl | 0.40 | g/l KCl | 0.40 | g/l KCl |
| | | 0.61 | g/l Na₂HPO₄ | 0.048 | g/l | 0.048 | g/l Na₂HPO₄ | 0.14 | g/l Na₂HPO₄ |
| 2.5 | g/l KH₂PO₄ | 0.19 | g/l KH₂PO₄ | Na₂HPO₄ | | 0.060 | g/l KH₂PO₄ | | |
| | | | | 0.060 | g/l KH₂PO₄ | | | 0.2 | g/l CaCl₂ |
| | | | | 0.140 | g/l CaCl₂ | | | | |
| | | | | 0.100 | g/l MgCl₂ | | | 0.097 | g/l MgSO₄ |
| | | | | 0.100 | g/l MgSO₄ | 0.352 | g/l NaHCO₃ | 1.5 | g/l NaHCO₃ |
| 17 | g/l casein peptone | | | 0.352 | g/l NaHCO₃ | | | | |

| 3 | g/l soya peptone | | | | | AA, Vitamins | |
|---|---|---|---|---|---|---|---|
| 2.5 | g/l glucose | 1.0 | g/l glucose | 1.0 | g/l glucose | 1.0 | g/l glucose |

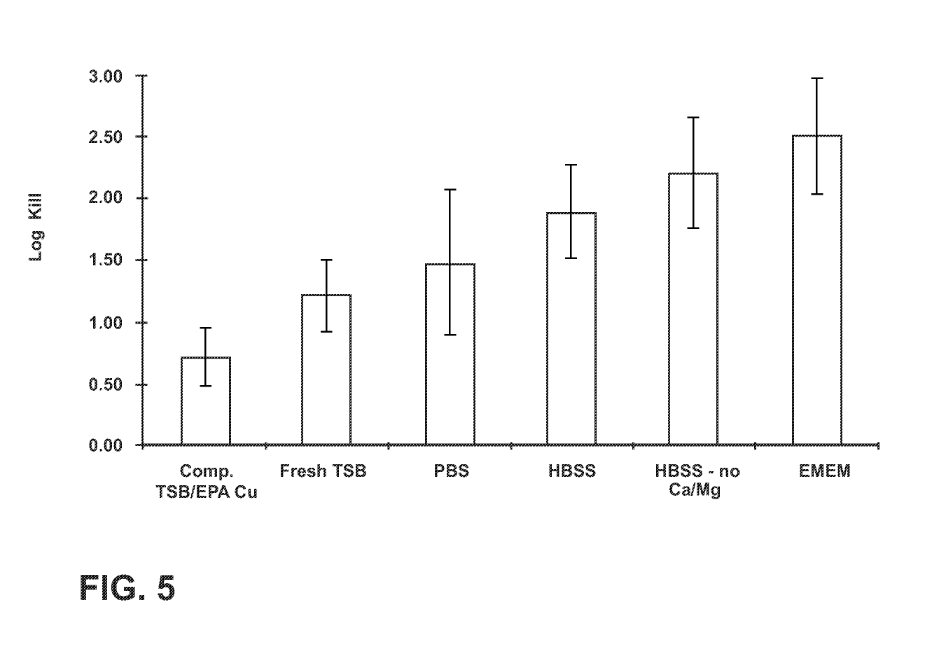

FIG. 5 depicts the results from an anti-microbial efficacy experiment conducted using the buffered test solutions 116 depicted in Table 1 according to the Ag Protocol 200 with respect to a test carrier 106 comprising an inorganic glass substrate having a silver-containing surface region. For comparison purposes, FIG. 5 also depicts anti-microbial testing results for conducting the Ag Protocol 200 in a way that mimics copper test protocol 100. In particular, the procedures for re-suspending bacterial colonies in fresh, buffered test solution 116 are bypassed with the use of copper test protocol 100. Hence, the inoculum 118 used for comparison purposes in FIG. 5 (“Comp. TSB/EPA Cu”) comprises a TSB solution, comparable to the “(A)” composition shown in FIG. 2.

As the results in FIG. 5 demonstrate, the log kill values for all but the Comp. TSB/EPA Cu group exceed 1. All the test solutions enabling an antimicrobial measurement>1 log kill have neutral pH and contain buffering systems. Only the Fresh TSB and the Comp. TSB/EPA Cu versions of the buffered test solution 116 depicted in FIG. 5 contain proteins. Hence, the other buffered test solutions 116 tested in this experiment and depicted in FIG. 5 do not contain proteins, reflecting the possible insight that proteins can detrimentally chelate with the silver ions in the surface region of the test carrier 106. Further, the PBS, HBSS, HBSS—no Ca/Mg and EMEM versions of the buffered test solution 116 possess no glucose or lower glucose levels compared to the TSB variants.

FIG. 5 demonstrates that the use of the HBSS and EMEM versions of buffered test solution 116 can achieve log kill values exceeding 2, significantly higher than the other versions of buffered test solution 116 tested. As such, buffered test solution 116 preferably comprises a solution comparable to HBSS, HBSS—no Ca/Mg and EMEM. These high log kill values associated with the HBSS and EMEM versions of buffered test solution 116 are indicative of high levels of anti-microbial testing efficacy for the Ag Protocol 200.

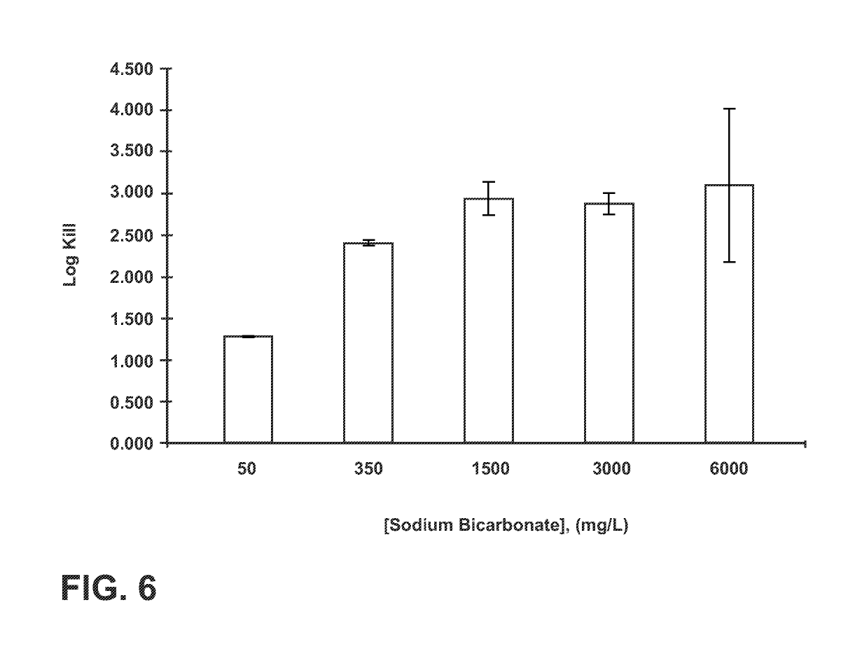

It should also be understood that only the HBSS and EMEM versions of buffered test solution 116 depicted in FIG. 5 and Table 1 contain sodium bicarbonate. The EMEM version of buffered test solution 116 had the highest log kill value, approaching an average of 2.5, and possessed a significantly higher level of sodium bicarbonate compared to HBSS and HBSS—no Ca/Mg. In view of these findings, another experiment was conducted to ascertain the effect of sodium bicarbonate in the buffered test solution 116 on the anti-microbial testing efficacy of a test carrier 106 having a silver-containing surface region. In this experiment, versions of buffered test solution 116 were created and used in tests according to the Ag Protocol 200 containing varying levels of sodium bicarbonate constituents. An HBSS solution was formulated comparably to the HBSS version depicted above in Table 1, but modified with the specified concentration levels of sodium bicarbonate ranging from 50 mg/1 to 6000 mg/l. As FIG. 6 demonstrates, log kill appears to correlate with concentration of sodium bicarbonate, with log kill values exceeding 2 for the HBSS buffered test solution 116 containing 350 mg/1 of sodium bicarbonate or more. As such, buffered test solution 116 should preferably contain sodium bicarbonate at a concentration of 350 mg/1 or more.

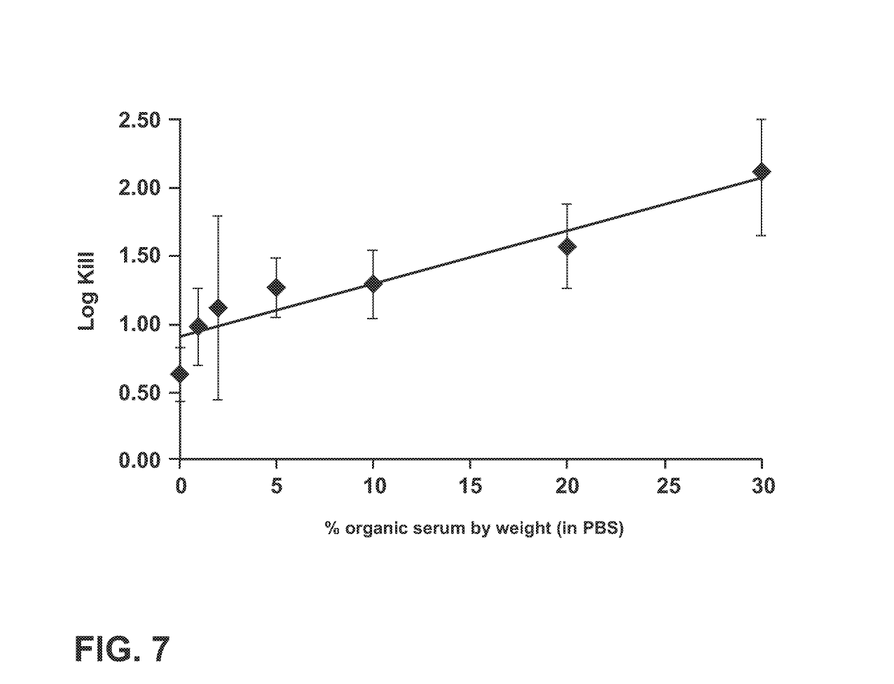

As noted earlier, organic serum 114 is added during step 150 of the Ag Protocol 200 for purposes of simulating soiling present on articles (e.g., electronic touch screen surfaces) employing the silver-containing surface region of test carrier 106. As depicted in FIG. 7, an increase in organic serum content within the inoculum 118 tends to increase measured log kill values. The experiment used to generate the results depicted in FIG. 7 was conducted according to the Ag Protocol 200 in a fashion similar to the experiments employed to generate the results depicted in FIGS. 5 and 6. In this experiment, however, the levels of organic serum 114 were varied during step 150 in the preparation of inoculum 118. Further, a PBS buffering solution 119 was employed in this experiment.

As FIG. 7 indicates, the increasing levels of organic serum tend to increase the log kill values. Organic serum levels as low as 10% tend to produce log kill values exceeding 1. Preferably, the organic serum 114 level employed in the Ag Protocol 200 is set at a concentration of 10 to 15% by weight. It is believed that higher organic serum 114 values that exceed 30%, while likely to produce even better log kill values, are not representative of the human “soil” encountered in the natural applications of the articles having silver-containing surface regions, such as those employed as test carrier 106. It should also be understood that limiting the organic serum 114 levels ensures that the Ag Protocol 200 is fairly comparable to the Copper Test Protocol, which typically relies on an organic serum concentration of 5% by weight (e.g., organic soil 14).

In another study, the Ag Protocol 200 and the copper test protocol 100 were employed to generate anti-microbial efficacy test results for test carriers 106 having a surface region containing silver ions and control test carriers 106a for four different bacterial organisms (e.g., bacterial organisms 110c developed at step 140). Consistent with the discussion above, a buffered test solution 116 was employed with a formulation comparable to EMEM for the Ag Protocol 200. The results from this work are listed below in Table 2. The table clearly demonstrates the superiority of the Ag Protocol 200 for testing the anti-microbial efficacy of articles having a surface region containing silver ions, particularly for Staphylococcus aureus and Escherichia coli.

TABLE 2

| Microorganism | Ag Protocol 200 | Copper Protocol 100 |
|---|---|---|
| S. aureus | 99.8% | 70% |
| E. aerogenes | 99.999% | 99.98% |
| P. aeruginosa | >99.999% | 99.4% |
| E. coli | 99.7% | 60% |

It will be apparent to those skilled in the art that various modifications and variations can be made without departing from the spirit or scope of the claims.

What is claimed is:

1. A method for testing the anti-microbial efficacy of a silver-containing surface region, comprising the steps:
inoculating nutrient agar with a portion of a stock having a plurality of bacterial organisms to form a culture;
incubating the culture to form a first incubated culture;
incubating a portion of the first incubated culture with nutrient agar to form a second incubated culture;
incubating a portion of the second incubated culture with nutrient agar to form a third incubated culture;
incubating the third incubated culture for approximately 48 hours to form an inoculated test plate with a plurality of bacterial colonies;
forming an inoculum by suspending a portion of the plurality of bacterial colonies in a buffered test solution, adjusting the test solution to a pH of approximately 7 to 8, and adding an organic soil serum at a concentration of approximately 10% to 30% by weight to the test solution;
inoculating a silver-containing surface region of a test carrier with a portion of the inoculum;
incubating the inoculated test carrier for at least approximately two hours;
washing the incubated and inoculated test carrier in a neutralizing solution to form a residual test inoculum;
counting the number of surviving bacterial colonies per volume in the residual test inoculum; and
calculating the percent reduction in the number of surviving bacterial colonies in the residual test inoculum relative to a residual control inoculum.

2. The method of claim 1, wherein the test carrier further comprises an inorganic glass material.

3. The method of claim 1, wherein the silver-containing surface region comprises a hydrophobic layer.

4. The method of claim 3, wherein the hydrophobic layer comprises a polymeric coating.

5. The method of claim 1, wherein the buffered test solution comprises a Hank's Balanced Salt Solution.

6. The method of claim 1, wherein the buffered test solution comprises a modified Hank's Balanced Salt Solution having substantially no calcium and magnesium ions.

7. The method of claim 1, wherein the buffered test solution comprises a phosphate buffered saline solution.

8. The method of claim 1, wherein the buffered test solution comprises a Minimal Essential Medium solution.

9. The method of claim 1, wherein the buffered test solution comprises a tryptic soy broth solution.

10. The method of claim 1, wherein the buffered test solution further comprises sodium bicarbonate at a concentration of at least 350 mg/l.

11. The method of claim 1, wherein the forming an inoculum step comprises adding the organic soil serum at a concentration of approximately 10% to 15% by weight.

12. The method of claim 1, wherein the incubating the inoculated test carrier step is conducted for at least approximately four hours.

13. A method for testing the anti-microbial efficacy of a silver-containing surface region, comprising the steps:
inoculating nutrient agar with a portion of a stock of bacterial organisms to form a culture;
incubating the culture to form a first incubated culture;
incubating a portion of the first incubated culture with nutrient agar to form a second incubated culture;
incubating a portion of the second incubated culture with nutrient agar to form a third incubated culture;
incubating the third incubated culture for approximately 48 hours to form an inoculated test plate with a plurality of bacterial colonies;
forming an inoculum by suspending a portion of the plurality of bacterial colonies in a buffered test solution, adjusting the test solution to a pH of approximately 7 to 8, and adding an organic soil serum at a concentration of approximately 10% to 30% by weight to the test solution;
inoculating a silver-containing surface region of a test carrier comprising an inorganic glass material with a portion of the inoculum;
incubating the inoculated test carrier for at least approximately two hours;
washing the incubated and inoculated test carrier in a neutralizing solution to form a residual test inoculum;
counting the number of surviving bacterial colonies per volume in the residual test inoculum; and
calculating the percent reduction in the number surviving bacterial colonies in the portion of the residual test inoculum relative to a residual control inoculum,
wherein the plurality of bacterial organisms is selected from one of the group consisting of Staphylococcus aureus, Enterobacter aerogenes, Pseudomonas aeruginosa and Escherichia coli.

14. The method of claim 13, wherein the silver-containing surface region comprises a hydrophobic layer.

15. The method of claim 14, wherein the hydrophobic layer comprises a polymeric coating.

16. The method of claim 13, wherein the buffered test solution comprises a Hank's Balanced Salt Solution.

17. The method of claim 13, wherein the buffered test solution comprises a modified Hank's Balanced Salt Solution having substantially no calcium and magnesium ions.

18. The method of claim 13, wherein the buffered test solution comprises a phosphate buffered saline solution.

19. The method of claim 13, wherein the buffered test solution comprises a Minimal Essential Medium solution.

20. The method of claim 13, wherein the buffered test solution comprises a tryptic soy broth solution.

21. The method of claim 13, wherein the buffered test solution further comprises sodium bicarbonate at a concentration of at least 350 mg/l.

22. The method of claim 13, wherein the forming an inoculum step comprises adding the organic soil serum at a concentration of approximately 10% to 15% by weight.

23. The method of claim 13, wherein the incubating the inoculated test carrier step is conducted for at least approximately four hours.

24. A method of preparing an inoculum for testing the anti-microbial efficacy of a silver-containing surface region, comprising the steps:
inoculating nutrient agar with a portion of a stock of bacterial organisms to form a culture;
incubating the culture to form a first incubated culture;
incubating a portion of the first incubated culture with nutrient agar to form a second incubated culture;
incubating a portion of the second incubated culture with nutrient agar to form a third incubated culture;
incubating the third incubated culture for approximately 48 hours to form an inoculated test plate with a plurality of bacterial colonies; and
forming an inoculum by suspending a portion of the plurality of bacterial colonies in a buffered test solution, adjusting the test solution to a pH of approximately 7 to 8, and adding an organic soil serum at a concentration of approximately 10% to 30% by weight to the test solution,
wherein the plurality of bacterial organisms is selected from one of the group consisting of Staphylococcus aureus, Enterobacter aerogenes, Pseudomonas aeruginosa and Escherichia coli.

25. The method of claim 24, wherein the buffered test solution comprises a Hank's Balanced Salt Solution.

26. The method of claim 24, wherein the buffered test solution comprises a modified Hank's Balanced Salt Solution having substantially no calcium and magnesium ions.

27. The method of claim 24, wherein the buffered test solution comprises a phosphate buffered saline solution.

28. The method of claim 24, wherein the buffered test solution comprises a Minimal Essential Medium solution.

29. The method of claim 24, wherein the buffered test solution comprises a tryptic soy broth solution.

30. The method of claim 24, wherein the buffered test solution further comprises sodium bicarbonate at a concentration of at least 350 mg/l.

31. An inoculum for testing the anti-microbial efficacy of a silver-containing surface region, comprising:
an inoculum comprising (a) a plurality of bacterial colonies; (b) an organic soil serum at a concentration of approximately 10% to 15% by weight; and (c) a buffering solution,
wherein the inoculum has a pH of approximately 7 to 8.

32. The inoculum of claim 31, wherein the inoculum consists essentially of (a) an incubated test solution having a plurality of bacterial colonies; (b) an organic soil serum at a concentration of approximately 10% to 15% by weight; and (c) a buffering solution.

33. The inoculum of claim 31, wherein the plurality of bacterial colonies is cultured from one of the group of bacterial organisms consisting of Staphylococcus aureus, Enterobacter aerogenes, Pseudomonas aeruginosa and Escherichia coli.

34. The inoculum of claim 31, wherein the buffering solution comprises a Hank's Balanced Salt Solution.

35. The inoculum of claim 31, wherein the buffering solution comprises a modified Hank's Balanced Salt Solution having substantially no calcium and magnesium ions.

36. The inoculum of claim 31, wherein the buffering solution comprises a phosphate buffered saline solution.

37. The inoculum of claim 31, wherein the buffering solution comprises a Minimal Essential Medium solution.

38. The inoculum of claim 31, wherein the buffering solution comprises a tryptic soy broth solution.

39. The inoculum of claim 31, wherein the inoculum further comprises sodium bicarbonate at a concentration of at least 350 mg/l.

40. An antimicrobial glass comprising:
a glass substrate having a silver-containing surface region, wherein the surface region is characterized by a log kill rate of 2 or greater as tested by the inoculum of claim 31.

41. An antimicrobial glass comprising:
a glass substrate having a silver-containing surface region, wherein the surface region is characterized by a log kill rate of 2 or greater as tested by the method of claim 1.

42. An antimicrobial glass comprising:
a glass substrate having a silver-containing surface region, wherein the surface region is characterized by a log kill rate of 2 or greater as tested by the method of claim 13.

43. An antimicrobial glass comprising:
a glass substrate having a silver-containing surface region, wherein the surface region is characterized by a log kill rate of 2 or greater as tested by the method of claim 24.
